(19) United States
(12) Patent Application Publication
(10) Pub. No.: US 2014/0370541 A1
(43) Pub. Date: Dec. 18, 2014

(54) CELL CULTURE LIQUID/GAS-PHASE CHAMBER DEVICES AND RELATED METHODS, AND EXEMPLARY USE FOR MEASURING ACCUMULATION OF GAS-PHASE NITRIC OXIDE IN DIFFERENTIATED CULTURES OF NORMAL AND CYSTIC FIBROSIS AIRWAY EPITHELIAL CELLS

(71) Applicant: UNIVERSITY OF NORTH CAROLINA, Chapel Hill, NC (US)

(72) Inventors: Milan Joseph Hazucha, Chapel Hill, NC (US); Johnny Lee Carson, Chapel Hill, NC (US); Lawrence Edward Ostrowski, Durham, NC (US)

(73) Assignee: UNIVERSITY OF NORTH CAROLINA, Chapel Hill, NC (US)

(21) Appl. No.: 14/371,341

(22) PCT Filed: Jan. 9, 2013
(86) PCT No.: PCT/US2013/020789

§ 371 Date: Jul. 9, 2014

Related U.S. Application Data
(60) Provisional application No. 61/584,356, filed on Jan. 9, 2012.

(51) Int. Cl. G01N 33/50; C12M 1/34; C12M 1/42; C12M 1/107; C12P 3/00

(52) U.S. Cl. CPC G01N 33/5038; C12M 21/04; C12P 3/00; C12M 35/08; C12M 41/34; G01N 2033/4977
USPC 435/34; 435/300.1; 435/168

(57) ABSTRACT

Embodiments disclosed herein include cell culture liquid/gas-phase chamber devices and related methods that allow measurement of gas-phase components and their production rate as well as substances/mediators released into media by cultured cells in response to various stimuli. In embodiments disclosed herein, these chamber devices are cell culture liquid/gas-phase chamber devices. The gas and liquid phase compartments are separated by the cell culture liquid/gas-phase chamber devices so that there is no, or minimal, physico-chemical interaction between the liquid and gas phase compartments. As an example, these cell culture liquid/gas-phase chamber devices can be used to study the effects of various pharmacologic agents or other interventions on healthy or diseased cell metabolism by assessing a release of metabolic products in liquid (culture media) and the headspace under standardized conditions. The reusable cell culture chamber devices allow for studying cultured cell responses under a short or prolonged incubation time.

### PRIORITY APPLICATIONS

The present application claims priority to PCT Patent Application Serial No. PCT/US2013/020789 filed on Jan. 9, 2013 entitled “Cell Culture Liquid/Gas-Phase Chamber Devices And Related Methods, and Exemplary Use For Measuring Accumulation Of Gas-Phase Nitric Oxide In Differentiated Cultures Of Normal And Cystic Fibrosis Airway Epithelial Cells;” which claims priority to U.S. Provisional Patent Application Ser. No. 61/584,356 filed on Jan. 9, 2012 and entitled “Two Phase Cell Culture Chambers, and Exemplary Use For Measuring Accumulation of Gas-Phase Nitric Oxide In Differentiated Cultures Of Normal And Cystic Fibrosis Epithelial Cells,” both of which are incorporated herein by reference in their entireties.

### GOVERNMENT LICENSE RIGHTS

This invention was made with U.S. Government support under grant number R01HL071798 awarded by the NIH National Heart Lung and Blood Institute. Thus, the U.S. Government has certain rights in this invention.

### REFERENCES

A references addendum is enclosed herewith and forms part of the present application and is incorporated herein by reference in its entirety. Citations to these references herein are noted in parenthesis.

### BACKGROUND

Nitric oxide (NO) is produced by nitric oxide synthase (NOS) through the enzymatic oxidation of L-arginine. Three different isoforms of NOS have been identified; nNOS(NOS1) and eNOS(NOS3) are described as being constitutively expressed and regulated by calcium, whereas iNOS(NOS2) is constitutively active and can be induced to high levels of expression in response to certain stimuli. All three enzymes have been reported to be expressed in the pulmonary system, with different isoforms expressed in different cell types (reviewed in (1)). For example, nNOS has been reported to be expressed in nerve fibers in airway smooth muscle, while eNOS is found in endothelial cells of blood vessels. Intriguingly, eNOS has also been reported to be localized to the base of cilia in rat airway epithelial cells (2), where it may play a role in the regulation of ciliary beat frequency (3-5). Expression of iNOS has been reported in numerous cell types, including airway epithelial cells and neutrophils. NO can have many different functions in the airways and depending on the level of NO produced and the site of release can act as a bronchodilator, a vasodilator, an antimicrobial, and a pro-inflammatory molecule.

The level of NO, measured in the gas phase, from either the lower airways as exhaled NO (eNO) or from the upper airways as nasal NO (nNO), has been shown to be altered in several disease states. In asthma, the level of eNO is increased compared to normal, and the level of NO has been shown to correlate with the level of inflammation (1, 6). The increase in NO is believed to be produced by the action of iNOS, which is induced to high levels in airway epithelium by pro-inflammatory cytokines, including IL-13 and IFN-γ (7-10). In contrast, the level of nNO in patients with primary ciliary dyskinesia (PCD) is drastically reduced compared to the levels observed in normal patients, and this finding is so consistent that the measurement of nNO is now being used as an aid to diagnosis (11-15). However, the mechanism responsible for the low levels of nNO in PCD has not yet been identified. In cystic fibrosis (CF), a disease characterized by chronic infection and inflammation, the level of eNO and nNO have also been observed to be lower than in normal controls, although the levels vary widely and are generally higher than those observed in PCD patients (16-18). The low level of NO in CF patients in the presence of chronic inflammation is not completely understood. In vitro studies have compared NO production between CF and control cells using various transformed cell lines grown in submerged culture. However, it is unclear if the regulation of NO production in these undifferentiated cells is representative of in vivo conditions (19, 20). Further, none of the prior studies have actually measured the level of NO released into the gas-phase. Recently, Suresh et al. described a method for measuring the gas-phase release of NO by cultured airway epithelial cells (21).

### SUMMARY OF THE DETAILED DESCRIPTION

Embodiments disclosed herein include cell culture liquid/gas-phase chamber devices and related methods that allow measurement of gas-phase components and their production rate, as well as substances/mediators released into media by cultured cells in response to various stimuli. In embodiments disclosed herein, these chamber devices are cell culture liquid/gas-phase chamber devices. Air and liquid phase compartments are separated by the cell culture liquid/gas-phase chamber devices so that there is no, or minimal, physico-chemical interaction between the liquid and gas phase compartments. As an example, these cell culture liquid/gas-phase chamber devices can be used to study the effects of various pharmacologic agents or other interventions on healthy or diseased cell metabolism by assessing a release of metabolic products in liquid (culture media) and the headspace under standardized conditions. The reusable chamber devices allow for studying cultured cell responses under a short or prolonged incubation time. As a non-limiting example, the cell culture liquid/gas-phase chamber devices may be used to measure accumulation of gas-phase Nitric Oxide (NO) in differentiated cultures of normal and cystic fibrosis airway epithelial cells.

For example, in one embodiment, a liquid/gas-phase chamber device for producing gas-phase and liquid-phase components released by cultured cells in response to stimuli is provided. The liquid/gas-phase chamber device comprises a chamber body forming an internal chamber. The liquid/gas-phase chamber device also comprises an air-liquid partition disposed in the internal chamber configured to interactively separate the internal chamber into a liquid compartment and a gas compartment. The liquid/gas-phase chamber device also comprises at least one transwell disposed through the air-liquid partition, and at least one transwell configured to support at least one cell culture. The at least one transwell comprises a first end disposed in the liquid compartment of the chamber body to capture a release of metabolic products from the cultured cells of the at least one cell culture in liquid disposed in the liquid compartment. The at least one transwell further comprises a second end disposed in the gas compartment of the internal chamber of the chamber body to capture a release of gas by the cultured cells of the at least one cell culture.

In this regard, in embodiments disclosed herein, the liquid/gas-phase chamber devices disclosed herein can be used to detect the level of gas-phase NO (gNO) in the airspace above primary cultures of control human nasal and bronchial epithelial cells and CF human bronchial epithelial (HBE) cells under several different conditions. The results demonstrate that primary cultures of healthy nasal epithelial cells produce measureable amounts of gNO. The results also demonstrate that well-differentiated cultures of bronchial epithelial cells can be stimulated with IFN-γ to accumulate large amounts of gNO. The results further demonstrate that under these conditions, the level of gNO in CF cultures is lower than the level in control cultures, and therefore this system can be used to further investigate the mechanisms responsible for the observed low levels of eNO in CF patients. A greater understanding of the regulation of NO production and its many functions may lead to new therapeutic approaches to CF, asthma, PCD and other pulmonary diseases.

In this regard, in another embodiment, a method of producing gas-phase component releases of cultured cells in response to stimuli is provided. The method comprises transferring at least one transwell containing cultured cells from at least one cultured cell growth tray to an air-liquid partition for a liquid/gas-phase chamber device. The method also comprises disposing stimuli liquid into a liquid compartment of an interior chamber of a chamber body of the liquid/gas-phase chamber device formed by the air-liquid partition disposed in an internal chamber of the chamber body the air-liquid partition configured to interactively separate the internal chamber into the liquid compartment and a gas compartment. The method also comprises inserting the air-liquid partition with the at least one transwell disposed therein into the internal chamber of the chamber body to place at least one membrane of the at least one transwell in contact with the stimuli liquid disposed in the liquid compartment of the internal chamber of the chamber body. The method also comprises sealing the internal chamber with a lid received by a top portion of the chamber body forming at least a portion of the gas compartment of the internal chamber to provide an air-tight chamber body. The method also comprises closing a first valve in fluid contact with the gas compartment of the internal chamber of the chamber body, and a second valve in fluid contact with the gas compartment of the internal chamber of the chamber body. The method also comprises incubating the cultured cells in the sealed internal chamber of the sealed chamber body for a defined period of time to allow the cultured cells to release a metabolic product in the stimuli liquid in the liquid compartment and release a metabolic product in the gas compartment, in response to exposure of the cultured cells to the stimuli liquid.

In one example, human bronchial epithelial (HBE) cells from CF and control tissues were cultured under ALI conditions that promote differentiation into a mostly ciliated, pseudo-stratified epithelium similar to that of the in vivo airway. Cultures were incubated in gas-tight chambers and the concentration of gNO was measured using a Sievers nitric oxide analyzer.

In CF and control cultures (both nasal and bronchial), the level of accumulated gNO under baseline conditions was low (<20 ppb). Treatment with interferon gamma (IFN-γ) induced iNOS expression and increased gNO significantly in differentiated cultures, while having no significant effect on undifferentiated cultures. Submersion of the apical surface with fluid drastically reduced the level of gNO. Importantly, the average level of gNO measured after IFN-γ treatment of control cells (576 ppb) was 3-fold greater than that from CF cells (192 ppb).

The exemplary results demonstrated that the lower level of exhaled NO observed in CF patients is reproduced in well-differentiated primary cultures of HBE cells treated with IFN-γ, supporting the hypothesis that the regulation of NO production is altered in CF. The results also demonstrate that IFN-γ treatment of differentiated cells results in higher levels of gNO than treatment of undifferentiated cells, and that a layer of fluid on the apical surface drastically reduces the amount of gNO, possibly by limiting the availability of oxygen.

### BRIEF DESCRIPTION OF THE FIGURES

FIG. 1A is a micrograph of an early stage culture of HBE cells consisting of a mostly single-cell layer of undifferentiated cells;

FIG. 1B is a micrograph of differentiated cultures consisting of a pseudo-stratified epithelium stained with hematoxylin and eosin with abundant ciliated cells at the apical surface;

FIG. 2A is a schematic diagram of an exemplary two-phase, liquid/gas-phase cell culture chamber device developed for the measurement of gNO or other components of a gas phase;

FIG. 2B is a schematic diagram of the exemplary cell culture liquid/gas-phase chamber device in FIG. 2A in assembled form provided as a modified Teflon® jar with manufactured components built to function as cell culture liquid/gas-phase chamber for the measurement of gNO (assembled);

FIG. 2C is a schematic diagram of the two-phase cell culture liquid/gas-phase chamber device in FIG. 2A in disassembled form with manufactured components built to function as cell culture liquid/gas-phase chamber for the measurement of gNO;

FIG. 3 is a flowchart illustrating an exemplary process for employing the two-phase cell culture liquid/gas-phase chamber device in FIGS. 2A-2C to detect the level of gas-phase NO (gNO) in the air space above primary cultures disposed in the two-phase cell culture liquid/gas-phase chamber device and other exemplary processes;

FIG. 4A is a preliminary experiment using two-phase cell culture liquid/gas-phase chamber device in FIGS. 2A-2C, showing the increase in gNO following IFN-γ treatment and the inhibition of gNO production by L-NMMA in an actual experimental trace from NO analyzer, where three (3) separate cultures were measured;

FIG. 4B shows an actual trace from a NO analyzer coupled to the two-phase cell culture liquid/gas-phase chamber device in FIGS. 2A-2C under the following conditions: A: Control measurement in HBSS medium after 6 hrs in the collection system. B: The same cells after 6 hr incubation with 10 μL INF-γ (100 ng/ml) in the medium illustrating marked elevation in NO concentration. C: The same cell culture incubated in the vessel for 6 hr 22 hrs post stimulation with INF-γ reflecting extended production of NO. Arrow denotes cessation of washout. Data obtained on three transwells in a chamber per measurement;

FIG. 5 is a level of gas-phase NO in control cultures analyze the head space gas chamber of the two-phase cell culture liquid/gas-phase chamber in FIGS. 2A-2C, wherein differentiated (Diff) and undifferentiated (Undiff) cultures of HBE cells were incubated with or without 100 ng/ml of IFN-γ for 19-20 hours and the level of NO in the airspace above the cultures was measured. The baseline level of NO was very low in both differentiated and undifferentiated cultures, but was stimulated to high levels in differentiated cultures treated with IFN-γ;

FIG. 6 is an example of induction of iNOS by IFN-γ in control (Normal) and cystic fibrosis (CF) cultures of HBE cells, wherein well differentiated cultures of CF and control cells were incubated in a two-phase cell culture liquid/gas-phase chamber of FIGS. 2A-2C with the indicated concentration of IFN-γ (ng/ml), wherein RT-PCR was performed to qualitatively analyze the levels of iNOS mRNA, and with treatment with IFN-γ induced iNOS expression in both the CF and control cells;

FIG. 7 is an example of the level of gas-phase NO in cystic fibrosis (CF) cultures analyzed from a two-phase cell culture liquid/gas-phase chamber of FIGS. 2A-2C, wherein differentiated (Diff) and undifferentiated (Undiff) cultures of CF cells were incubated with or without 100 ng/ml of IFN-γ for 19-20 hours and the level of NO in the airspace above the cultures was measured, and where the baseline level of gNO was low in differentiated cultures, but was stimulated to high levels in differentiated cultures treated with IFN-γ, and where levels of gNO were low in undifferentiated cultures with or without IFN-γ treatment;

FIG. 8 is an exemplary chart showing gas-phase NO levels from control and CF HBE cell cultures; and

FIG. 9 is an exemplary chart showing the correlation of gas-phase NO with NOx concentration in the basal media.

### DETAILED DESCRIPTION

Reference will now be made in detail to the embodiments, examples of which are illustrated in the accompanying drawings, in which some, but not all embodiments are shown. Indeed, the concepts may be embodied in many different forms and should not be construed as limiting herein; rather, these embodiments are provided so that this disclosure will satisfy applicable legal requirements. Whenever possible, like reference numbers will be used to refer to like components or parts.

Embodiments disclosed herein include cell culture liquid/gas-phase chamber devices and related methods that allow measurement of gas-phase components and their production rate as well as substances/mediators released into media by cultured cells in response to various stimuli. In embodiments disclosed herein, these chamber devices are cell culture liquid/gas-phase chamber devices. The air and liquid phase compartments are separated by the cell culture liquid/gas-phase chamber devices so that there is no or minimal physico-chemical interaction between the liquid and gas phase compartments. As an example, these cell culture liquid/gas-phase chamber devices can be used to study the effects of various pharmacologic agents or other interventions on healthy or diseased cell metabolism by assessing a release of metabolic products in liquid (culture media) and the headspace under standardized conditions. The reusable chamber devices allow for studying cultured cell responses under a short or prolonged incubation time. As a non-limiting example, the cell culture liquid/gas-phase chamber devices may be used to measure accumulation of gas-phase Nitric Oxide (NO) in differentiated cultures of normal and cystic fibrosis airway epithelial cells.

For example, in one embodiment, a liquid/gas-phase chamber device for producing gas-phase and liquid-phase components released by cultured cells in response to stimuli is provided. The liquid/gas-phase chamber device comprises a chamber body forming an internal chamber. The liquid/gas-phase chamber device also comprises an air-liquid partition disposed in the internal chamber configured to interactively separate the internal chamber into a liquid compartment and a gas compartment. The liquid/gas-phase chamber device also comprises at least one transwell disposed through the air-liquid partition, and at least one transwell configured to support at least one cell culture. The at least one transwell comprises a first end disposed in the liquid compartment of the chamber body to capture a release of metabolic products from the cultured cells of the at least one cell culture in liquid disposed in the liquid compartment. The at least one transwell further comprises a second end disposed in the gas compartment of the internal chamber of the chamber body to capture a release of gas by the cultured cells of the at least one cell culture.

As will be discussed in more detail below, in embodiments disclosed herein, the liquid/gas-phase chamber devices can be used to detect the level of gas-phase NO (gNO) in the airspace above primary cultures of control and CF human bronchial epithelial (HBE) cells under several different conditions. The results demonstrate that well-differentiated cultures of airway epithelial cells can be stimulated with IFN-γ to accumulate large amounts of gNO. The results further demonstrate that under these conditions, the level of gNO in CF cultures is lower than the level in control cultures, and therefore this system can be used to further investigate the mechanisms responsible for the observed low levels of eNO in CF patients. A greater understanding of the regulation of NO production and its many functions may lead to new therapeutic approaches to CF, asthma, PCD and other pulmonary diseases.

### Measurement of Gas-Phase NO

In preliminary studies, attempts were made to measure gNO release from well-differentiated cultures of HBE cells under the conditions described by Suresh et al. (21). Because some difficulties were encountered in obtaining a reproducible, gas-tight seal, both around the holes drilled in the plastic for the fittings and around the lid of the dish using parafilm, the procedure was modified in the following manner. First, 120 ml Teflon® jars (Savillex) were fitted with silicone O-rings to create a leak-proof seal. Two holes were drilled in the lid and fitted with stainless steel luer-lock adapters that were fastened tightly using stainless steel washers and nuts. The luer-lock adapters were fitted with two 3-way plastic stop-cocks to allow connections to be made to the Sievers 270B NO or other analyzers and room or zero air. Manufactured Teflon partition was inserted into the jar to hold the transwells containing cells and to separate the liquid compartment from the headspace.

For measurement of gNO production by HBE cultures, ALI media was added to each chamber. The apical surface of each culture was washed with 1 ml of PBS for 5 minutes at 37° C. to remove mucus and cell debris. Unless otherwise specified, 50 μl of PBS was added to the apical surface of the culture to provide a thin layer of ASL and allow cilia to beat freely. Three, 12 mm cultures were placed in each chamber, and the chambers were placed in a 37° C./5% CO₂ incubator with the lids removed for 5 minutes to allow equilibration with incubator air. The lids were then replaced and tightened with both stop-cocks fully closed. After the appropriate incubation time, the chambers were removed from the incubator and connected to an NO analyzer. The stop-cocks were simultaneously opened to allow room air into the chamber while sample was being withdrawn into the analyzer for measurement. For these studies, NO was measured using a Sievers 280B nitric oxide analyzer with a flow rate of 40 ml/min. For the measurements of gNO production by nasal cultures the flow rate was increased to 500 ml/min. The analyzer was routinely calibrated using NO free air and nitric oxide standards, and zeroed before each experimental run. The level of NO was determined in room air and was usually <5 ppb. The analog output from the NO analyzer was directed into a MACLAB analog-digital converter and computer for data analyses and archiving. All NO measurements are reported as the peak concentration obtained during the sampling period, usually within the first 15 seconds.

Using the above conditions, several preliminary experiments were performed, including incubating the media filled chambers alone, to confirm the lack of NO release, submerging the chambers in a water-filled vessel to verify that there were no leaks, and filling the chambers with known concentrations of NO gas and measuring the amount of NO recovered. The amount of NO released from the same cultures in different chambers was also measured, with variable incubation times and performed repeated measures of the same cells under the same conditions. These studies demonstrated that the chambers provided a reproducible means to measure gNO production.

### Measurement of Total Nitrate/Nitrite

Measurements of total nitrite/nitrate in apical and basolateral media samples were performed using the Parameter kit (R&D Systems, Minneapolis, Minn.) according to the manufacturer's instructions. Briefly, a 0.5 ml sample of media was obtained from the basolateral chamber at the conclusion of the experiment and frozen at −20° C. until analyzed. Each sample was assayed in duplicate and compared to a standard curve, which was prepared in the same ALI media used to culture the cells.

### Statistical Analysis

Unless otherwise stated, all data is reported as mean+/−standard error of the mean. Usually, 2-3 replicate cultures from each donor were measured under each condition and the average value was obtained. The total number of cultures measured (n) and the number of individual donors is reported. Because the data was not normally distributed, the logarithm of the average NO concentration was used for statistical analysis (22). For comparison between the same donor cells under two conditions, a paired t-test was used; for all other comparisons, a one-tailed t-test assuming unequal variance was used. Results with a p<0.05 were considered significant.

### Results

### Culture of Cells and Measurement of Gas Phase NO

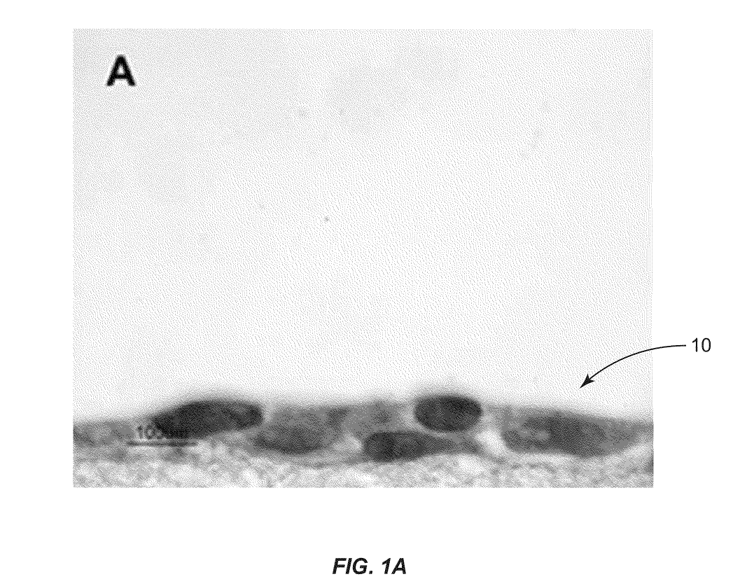

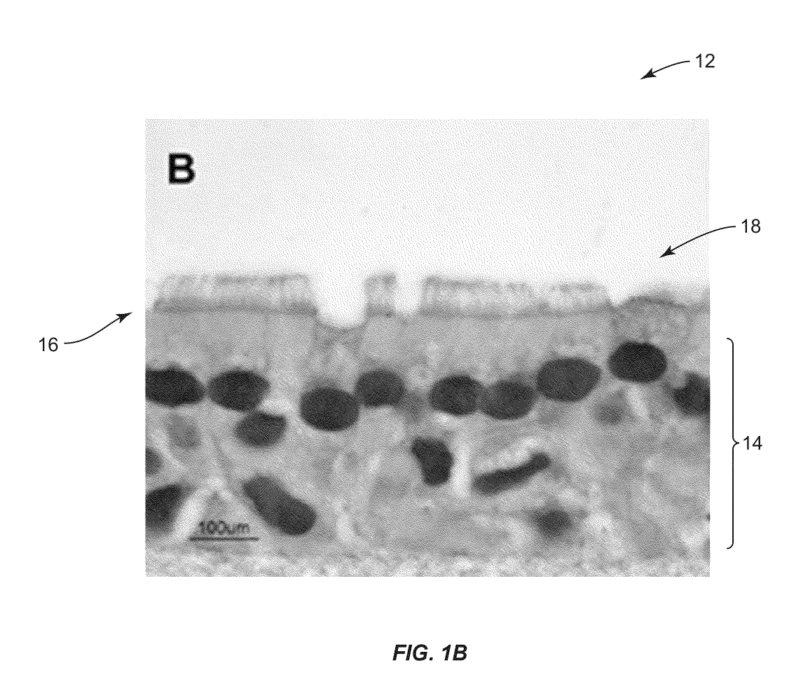

For the studies reported here, human bronchial epithelial (HBE) cells that had been passaged once were plated on collagen coated Millicell® membranes and after reaching confluence, were maintained at an air/liquid interface (ALI) for the duration of the experiment. Initially, the cultures consist of a single layer of undifferentiated cells 10, as illustrated in FIG. 1A. FIG. 1A is a micrograph of an early stage culture of HBE cells 10 consisting of a mostly single-cell layer of undifferentiated cells;

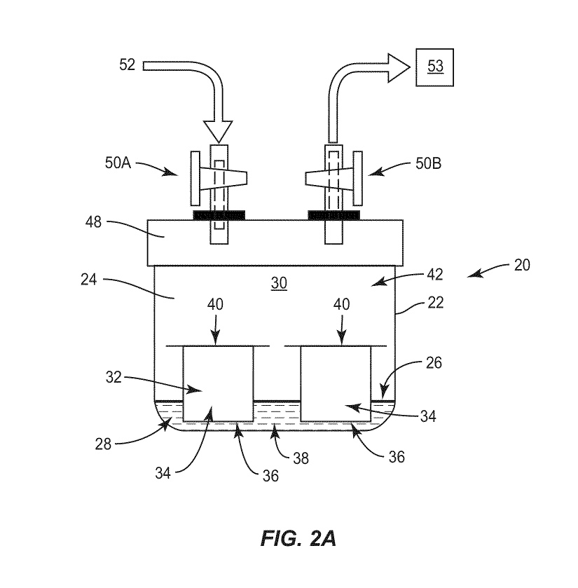

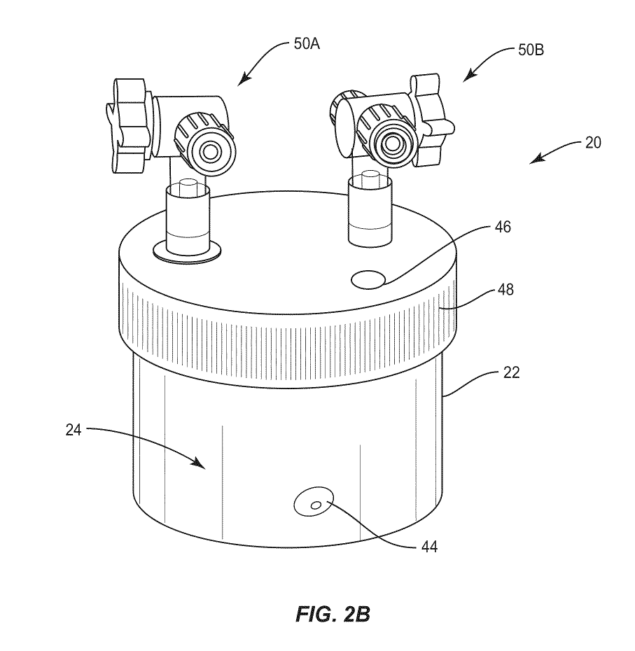

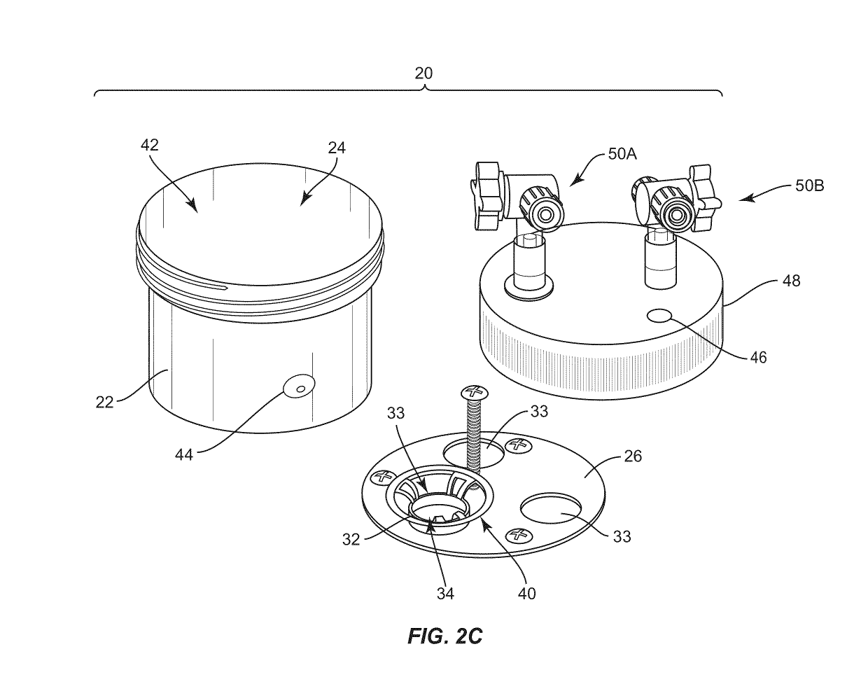

Ciliated cells first become visible ˜14 days after establishment of an air-liquid interface, and the number of ciliated cells continues to increase with time so that by ˜31 days, the cultures typically consist of a multi-layered, pseudo-stratified, heavily ciliated epithelium, as illustrated in FIG. 1B. FIG. 1B is a micrograph 12 of differentiated cultures 14 consisting of a pseudo-stratified epithelium 16 stained with hematoxylin and eosin with abundant ciliated cells at the apical surface 18. To measure gas-phase NO (gNO) production, the apical surface of the cultures were washed, a small volume of PBS (50 ul) was added to wet the surface, and the cultures were incubated overnight (19-20 hours) in a gas-tight, NO inert, cell culture liquid/gas-phase chamber developed for the measurement of gNO (schematically shown in FIG. 2A).

In this regard, the cell culture liquid/gas-phase chamber device described herein starting at FIG. 2A allows measurement of gas-phase components and their production rate as well as substances/mediators released into media by cultured cells in response to various stimuli. The air and liquid compartments are separated so that there is no or minimal physico-chemical interaction between the two. The chamber device can be used to study the effects of various pharmacological agents or other interventions on healthy or diseased cell metabolism by assessing a release of metabolic products in liquid (culture media) and the headspace under standardized conditions. The reusable chamber device allows for studying cultured cell responses under a short or prolonged incubation time.

In this regard, FIGS. 2A and 2B illustrate an exemplary Teflon® cell culture liquid/gas-phase chamber device 20 (also referred to herein as “chamber device 20”) modified for the measurement of gNO, assembled and unassembled, respectively. The level of accumulated NO in the gas-phase was measured by connecting the chamber directly to a nitric oxide analyzer in this example. FIG. 2A is a schematic diagram of the exemplary two-phase, liquid/gas-phase cell culture chamber device 20 developed for the measurement of gNO or other components of a gas phase. FIG. 2B is a schematic diagram of the exemplary cell culture liquid/gas-phase chamber device 20 in FIG. 2A in assembled form provided as a modified Teflon® jar with manufactured components built to function as cell culture liquid/gas-phase chamber for the measurement of gNO. FIG. 2C is a schematic diagram of the two-phase cell culture liquid/gas-phase chamber device 20 in FIG. 2A in disassembled form with manufactured components built to function as cell culture liquid/gas-phase chamber for the measurement of gNO.

In this regard with regard to FIGS. 2A-2C, the chamber device 20 is provided. The chamber device 20 is configured to produce gas-phase and liquid-phase components released by cultured cells in response to stimuli. The chamber device 20 in this embodiment comprises a chamber body 22 forming an internal chamber 24. The chamber body 22 could be made out Teflon® or stainless steel, which do not react or\react minimally with NO, as non-limiting examples. The chamber device 20 also includes an air-liquid partition 26, as illustrated in FIGS. 2A and 2C. The air-liquid partition 26 is configured to be disposed in the internal chamber 24 and configured to interactively separate the internal chamber 24 into a liquid compartment 28 and a gas compartment 30, as illustrated in FIG. 2A. One or more transwells 32, configured to support at least one cell culture 34, are disposed through an opening 33 disposed in the air-liquid partition 26, as illustrated in FIGS. 2A and 2C. The transwells 32 comprise a first end 36 disposed in the liquid compartment 28 of the chamber body 22 to capture a release of metabolic products from the cell culture 34 for being exposed to stimuli liquid 38 disposed in the liquid compartment 28, as illustrated in FIGS. 2A and 2C. The transwells 32 further comprise a second end 40 disposed in the gas compartment 30 of the chamber body 22 to capture a release of gas by the cell cultures 34. In this manner, the chamber device 20 allows measurement of gas-phase components and their production rate as well as substances/mediators released into media by the cultured cells 34 in response to various stimuli liquid 38. The liquid and gas compartments 28, 30 are separated so that there is no, or minimal, physico-chemical interaction between the liquid and gas compartments 28, 30. The chamber device 20 can be used to study the effects of various pharmacological agents or other interventions on healthy or diseased cell metabolism by assessing a release of metabolic products in liquid (culture media) and the headspace under standardized conditions. The reusable chamber device 20 allows for studying cultured cell 34 responses under a short or prolonged incubation time.

With continuing reference to FIGS. 2A-2C, the chamber body 22 may be produced from Teflon® to minimize physico-chemical reactions between the metabolic gas components released by the cultured cells 34 and the chamber body 22. The chamber device 20 may also optionally comprise one or more ports 44, 46 disposed in the chamber body 22. The ports 44, 46 allow injection of a material into or extraction of a material from the internal chamber 24 of the chamber device 20. Port 44 is in fluid contact with the liquid compartment 28 of the chamber body 22 to allow injection into or extraction of the stimulus liquid 38 from the chamber body 22. For example, it may be desired to provide port 44 for access so that a lid 48 secured to the chamber body 22 to seal off the internal chamber 24 is not required to be removed to inject or extract material. The lid 48 can also be made out of Teflon®, stainless steel and/or the same material as used for the chamber body 22, as non-limiting examples. Port 46 is in gaseous contact with the gas compartment 30 to allow injection of material into or extraction of gas-phase components released by the cultured cells 34 as a result of their disposition in the air-liquid partition 26 and in contact with the stimulus liquid 38.

With continuing reference to FIGS. 2A-2C, one or more valves 50A, 50B can be provided and fluidly coupled to the gas compartment 30 to allow ingress and egress of gas into and from the gas compartment 30. The valves 50 may be provided in the form of stopcocks, which can be opened for gas access to the gas compartment 30 and closed to close off access to the gas compartment 30. For example, as discussed in more detail below, the valves 50A, 50B can be open as illustrated in FIG. 2A to allow ambient air 52 to ingress into the gas compartment 30 through valve 50A and the metabolic gas component released by the cell culture 34 to be egressed through the valve 50B. An analyzer 53, or measurement tool or device 53, can be fluidly coupled to the valve 50B to receive the metabolic gas component released by the cell culture 34 in the gas compartment 30 to be analyzed or measured. As non-limiting examples, one or both of the valves 50A, 50B may be two-way valves with an open and close position, or three-way valves 50A, 50B that have two open positions and a closed position, for example, if more than one analyzer device is desired to be fluidly coupled to the chamber device 20.

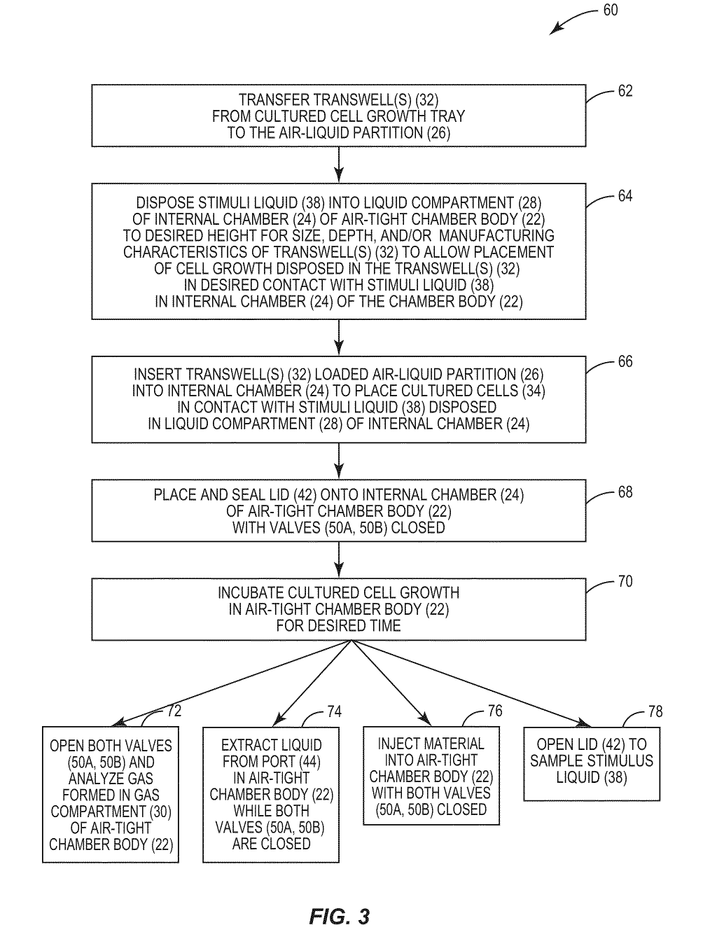

FIG. 3 is a flowchart illustrating an exemplary process 60 for employing the two-phase cell culture liquid/gas-phase chamber device 20 in FIGS. 2A-2C to detect the level of gas-phase NO (gNO) in the gas compartment 30 above primary cell cultures 34 disposed in the chamber device 20. In this regard, the process 60 in FIG. 3 is an exemplary method of producing gas-phase component releases of the cultured cells 34 in the chamber device 20 in FIGS. 2A-2C in response to the cultured cells 34 being placed into contact with the stimuli liquid 38. The method comprises transferring at least one transwell 32 containing the cultured cells 34 from at least one cultured cell growth tray to the air-liquid partition 26 for the chamber device 20 (block 62). The process then involves disposing the stimuli liquid 38 into the liquid compartment 28 of the internal chamber 24 of the chamber body 22 of the chamber device 20 formed by the air-liquid partition 26 disposed in the internal chamber 24 of the chamber body 22 (block 64). The air-liquid partition 26 is configured to interactively separate the internal chamber 24 into the gas compartment 30 and the liquid compartment 28, as previously described.

With continuing reference to FIG. 3, the process then involves inserting the air-liquid partition 26 with the transwell 32 disposed therein into the internal chamber 24 of the chamber body 22 to place the cell culture 34 in contact with the stimuli liquid 38 disposed in the liquid compartment 28 of the internal chamber 24 of the chamber body 22 (block 66). The process then involves sealing the internal chamber 24 of the chamber device 20 with the lid 42 received by a top portion of the chamber body 22 forming at least a portion of the gas compartment 30 of the internal chamber 24 to provide an air-tight chamber body 22 (block 68). The valves 50A, 50B are closed (block 68). Next, the process in FIG. 3 involves incubating the cultured cells 34 in the sealed internal chamber 24 of the sealed chamber body 22 for a defined period of time to allow the cultured cells 34 to release a metabolic product in the stimuli liquid 38 in the liquid compartment 28 and release a metabolic gas product in the gas compartment 30, in response to exposure of the cultured cells 34 to the stimuli liquid 38 (block 70).

With continuing reference to FIG. 3, the metabolic gas product can be analyzed or other optional steps performed. For example, both valves 50A, 50B could be opened as previously described and illustrated in FIG. 2A to allow ingress of ambient air 52 through valve 50A, and the egress of the metabolic gas component in the gas compartment 30 through valve 50B to be provided to the analyzer 53 (block 72). Alternatively, or in addition, portion of the stimuli liquid 38 could be extracted through port 44 illustrated in FIGS. 2A-2C and previously described, while both valves 50A, 50B are closed (block 74). Alternatively, or in addition, a material could be injected into the internal chamber 24, into the gas compartment 30 through port 46 or liquid compartment 28 through port 44, with both valves closed 50A, 50B (block 74). Alternatively, or in addition, the lid 42 could be opened and removed from the chamber body 22 to have greater access to the stimulus liquid 38 for sampling and analysis (block 78).

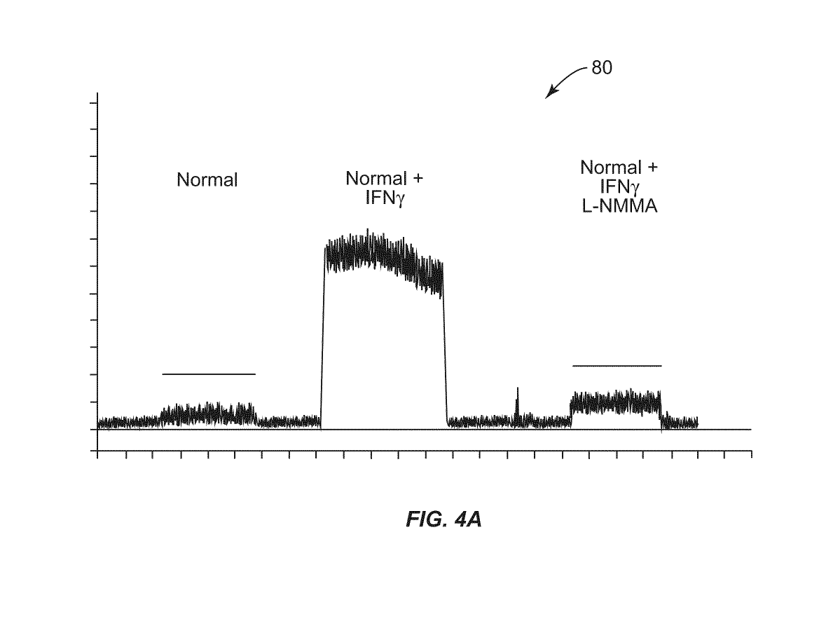

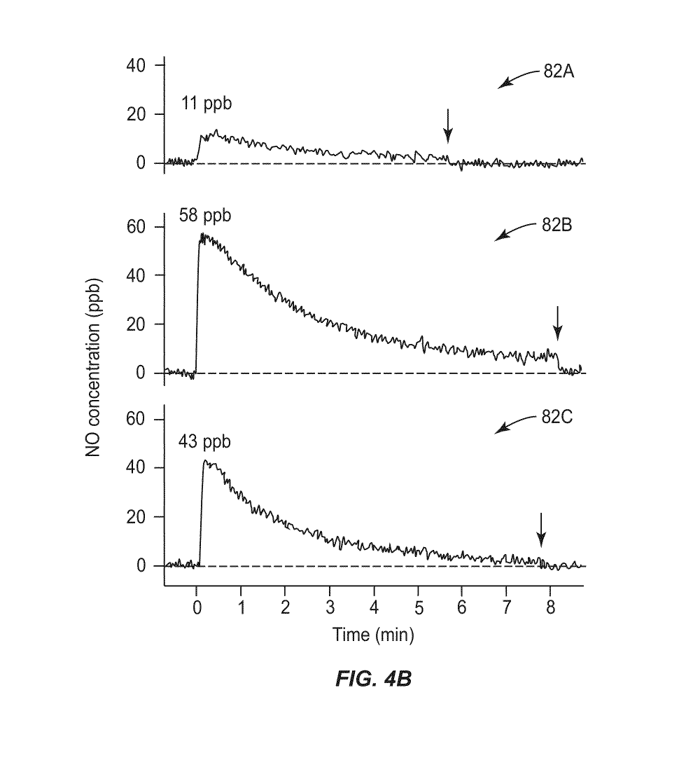

Preliminary studies showed that control HBE cells produced a low level of NO under baseline conditions. Replicate cultures from the same donor and repeated measures of the same cultures on consecutive days yielded similar values of NO, indicating that the technique was reproducible. Additional studies demonstrated that the low basal level of NO could be stimulated by treatment with interferon gamma (IFN-γ) and inhibited completely by L-NAME, a nitric oxide synthase inhibitor, as illustrated in the chart 80 in FIG. 4A. FIG. 4A is a preliminary experiment using two-phase cell culture liquid/gas-phase chamber 20 in FIGS. 2A-2C, showing the increase in gNO following IFN-γ treatment and the inhibition of gNO production by L-NMMA in an actual experimental trace from NO analyzer, where three (3) separate cultures were measured. The results in the chart 80 in FIG. 4A confirms that the NO measured was generated enzymatically by nitric oxide synthase (NOS) and that level of NO measured was responsive to experimental treatments. FIG. 4B shows an actual trace from a NO analyzer coupled to the two-phase cell culture liquid/gas-phase chamber 20 in FIGS. 2A-2C under the following conditions: 82A: Control measurement in HBSS medium after 6 hrs in the collection system; 82B: the same cells after 6 hr incubation with 10 μL INF-γ (100 ng/ml) in the medium illustrating marked elevation in NO concentration; and 82C: the same cell culture incubated in the vessel for 6 hr 22 hrs post stimulation with INF-γ reflecting extended production of NO. Arrow denotes cessation of washout. Data obtained on three transwells in a chamber per measurement.

An air-liquid partition 26, such as Teflon® air-liquid partition 26 in FIGS. 2A-2C, with openings 33 for cell culture transwells separates cell media from the air space above the cell culture 34. This two-compartment setup allows for measurement of both gaseous and liquid media substances/mediators released or consumed by cultured cells 34. The Teflon® air-liquid partition 26 prevents gas-liquid interaction, i.e., absorption of gases by the media or degassing from the media into a headspace. Typically, the current approach is to analyze only the media for substances of interest. The release of gases by cells at the apical surface is presently not measured since commercially available cell trays with transwells 32 are not constructed to support such measurements. In one embodiment, the lid 42 of the chamber device 20 in FIGS. 2A-2C has two fittings with 3-way valve in the form of valves to allow for a connection to a gas analyzer, to air or other desired gases and mixtures, e.g., oxygen to replace the withdrawn sample. One can also flush continuously or intermittently the head space and analyze the content or monitor continuously production of gases of interest over a prolonged period of time. This way the complete (air+media) metabolic and regulatory activity and mechanisms of actions of healthy or diseased cells can be studied independently.

In one embodiment, the chamber device 20 is a modified Teflon® jar with the air-liquid partition 26 and three (3) transwells 32 separating culture media from culture headspace or the gas compartment 30. This arrangement allows for studying cell metabolism separately in each compartment under standardized conditions. The chamber device can be taken apart and sterilized. Future improvements: (1) alteration of head space volume, (2) larger jar accommodating larger transwells, (3) additional ports installed on the side of a jar near the bottom (at media level) to allow for exchange of media, (4) add an injection port on the lid, (5) build-in glass window for viewing by microscope. The chamber device can used to investigate pathophysiology of other cell lines, e.g., kidney cells and their ciliary function, etc. Moreover, since this an airtight system it can be used to study the air-liquid interaction (with the partition removed) of media and gases.

### Gas-Phase NO Levels in Normal Human Bronchial Epithelial Cell Cultures

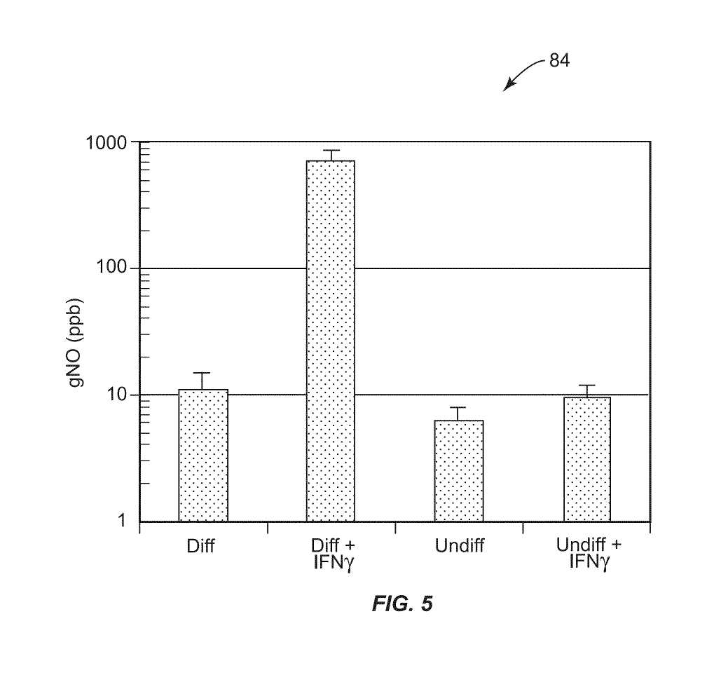

To begin to examine the regulation of NO production by human airway epithelial cells, the level of accumulated gNO in well-differentiated cultures of control HBE cells, grown under our standard conditions, was first measured (23). Levels of gNO were very low under baseline conditions in fully-differentiated cultures (mean age=100 days) averaging 10.9+/−5.1 ppb (n=20 from 7 donors) after overnight incubation, as illustrated in the chart 84 in FIG. 5. FIG. 5 is a level of gas-phase NO in control cultures analyze the head space gas chamber of the two-phase cell culture liquid/gas-phase chamber 20 in FIGS. 2A-2C, wherein differentiated (Diff) and undifferentiated (Undiff) cultures of HBE cells were incubated with or without 100 ng/ml of IFN-γ for 19-20 hours and the level of NO in the airspace above the cultures was measured. The baseline level of NO was very low in both differentiated and undifferentiated cultures, but was stimulated to high levels in differentiated cultures treated with IFN-γ.

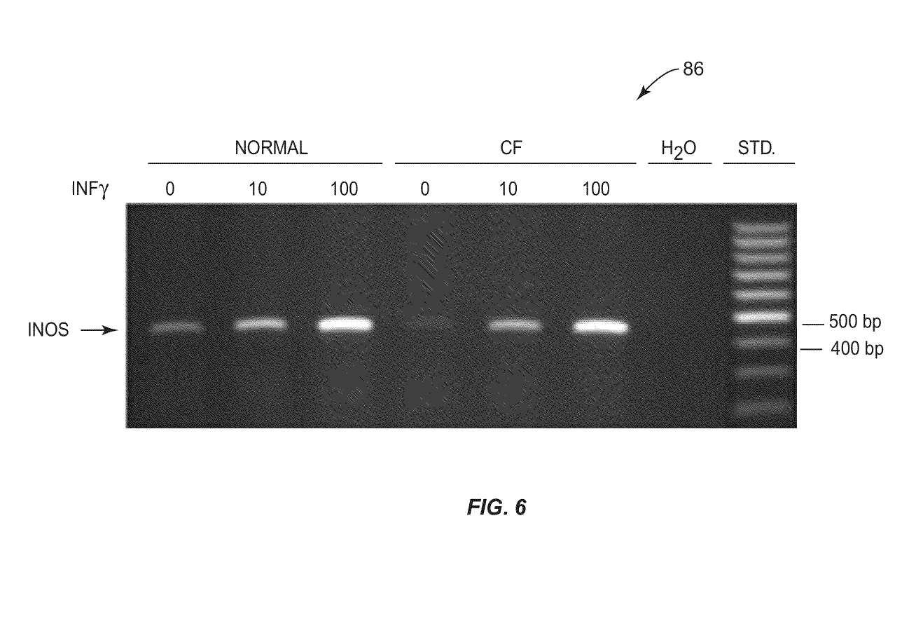

To examine the effect of an inflammatory cytokine on NO production, well-differentiated cultures of normal HBE cells were treated with different concentrations of interferon-gamma (IFN-γ), a potent inducer of iNOS (7-10). Total RNA was isolated and RT-PCR was used to qualitatively assess the level of the NOS isoforms. As expected, the level of iNOS RNA showed a clear increase with increasing concentrations of IFN-γ, as shown in the chart 86 in FIG. 6, and also increased with time of treatment (not shown). FIG. 6 is an example of induction of iNOS by IFN-γ in control (Normal) and cystic fibrosis (CF) cultures of HBE cells, wherein well differentiated cultures of CF and control cells were incubated in a two-phase cell culture liquid/gas-phase chamber 20 of FIGS. 2A-2C with the indicated concentration of IFN-γ (ng/ml), wherein RT-PCR was performed to qualitatively analyze the levels of iNOS mRNA, and with treatment with IFN-γ induced iNOS expression in both the CF and control cells. In contrast, the levels of nNOS and eNOS appeared relatively unchanged by treatment with IFN-γ (not shown). To examine the effect of IFN-γ on gNO, well-differentiated cultures (mean age 128 days) of control HBE cells were treated with interferon-gamma (IFN-γ) overnight. Treatment with 100 ng/ml of IFN-γ increased the level of accumulated NO>50-fold (see FIG. 5), averaging 576+/−200 ppb (n=22; 8 donors). The IFN-γ induced increase in NO was highly significant (p=6.5×10E-6). Increased levels of iNOS RNA and gNO were observed as early as 6 hours after treatment (data not shown), and levels of NO continued to increase for at least 24 hours.

Because almost all previous studies of NO production by airway epithelial cells have been performed on undifferentiated cells grown under submerged conditions or on immortalized cell lines, the level of gNO in cultures of undifferentiated HBE cells was examined. Levels of gNO under baseline conditions (mean age=8 days) was close to background in undifferentiated cultures, averaging 5.5+/−1.8 ppb (n=13 from 5 donors). This value is lower than that observed in differentiated cultures (10.9+/−5.1 ppb, above), but was not significantly different (see FIG. 5). The difference in gNO between undifferentiated and differentiated airway cells was more evident when cultures were treated with IFN-γ. Levels of gNO in undifferentiated cultures only increased slightly following IFN-γ treatment, to an average of 9.5+/−3.3 (n=12; 4 donors) compared to 5.5 ppb for the untreated cultures (see FIG. 5). The absence of a significant effect of IFN-γ treatment on gNO from undifferentiated cells is in sharp contrast to the ˜50-fold increase observed in differentiated cells.

Differentiated cultures of HBE cells produce and accumulate mucus and cellular debris on their apical surface between media changes (every 3-4 days). Because this layer of protein-rich airway surface liquid (ASL) has the potential to react with cellular NO, the apical surface of all cultures was routinely washed with PBS to remove accumulated mucus. As described in methods, the ASL was then replaced with a fixed amount of PBS (50 μL), which was sufficient to wet the surface and allow ciliary activity. In preliminary experiments, it was observed that larger volumes of apical fluid reduced the amount of gNO. To investigate the effect of fluid submersion in more detail, fully differentiated cultures of HBE cells were washed and 50 or 420 μL of PBS was added to the apical surface. Cultures were treated with IFN-γ and the gas-phase levels of NO were measured after 6 or 20 hours. The amount of NO was drastically reduced in cultures submerged for 6 hours with 420 μL of PBS, averaging only 6.6+/−5.6 ppb compared to 379+/−210 ppb in cultures incubated with 50 μL (n=5 cultures from 2 donors). The effect of submersion was maintained at longer times, with cultures measured after 20 hours averaging only 26.5+/−6.4 ppb compared to 1642.5+/−31 ppb (n=3 cultures from a single donor).

### Gas-Phase NO Levels in Cystic Fibrosis Airway Epithelial Cell Cultures

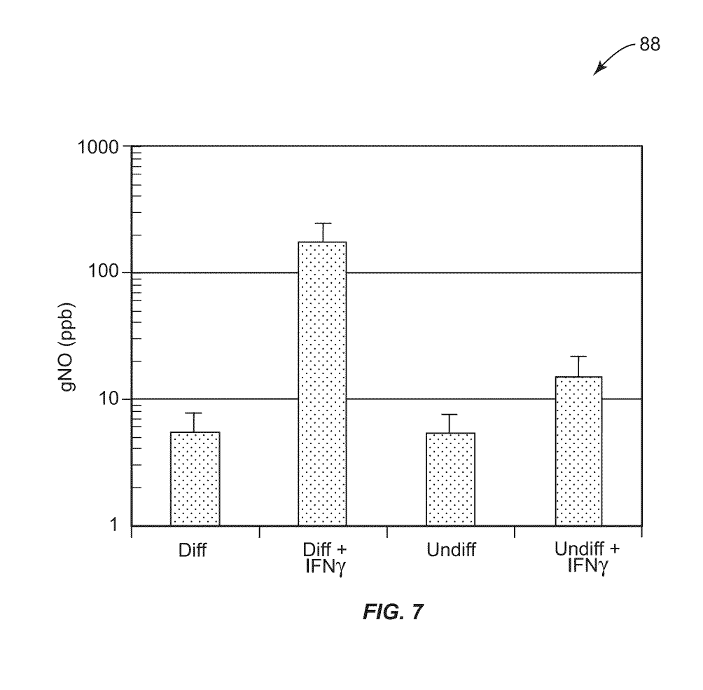

To determine if the lower levels of NO observed in CF patients would also be observed in vitro, HBE cells obtained from CF patients undergoing transplant were studied under the same conditions as above. As observed for the control cultures, the level of gNO at baseline was very low in CF cells, averaging only 5.8+/−2.5 ppb (n=14; 6 donors), as illustrated in the chart 88 in FIG. 7. FIG. 7 is an example of the level of gas-phase NO in cystic fibrosis (CF) cultures analyzed from a two-phase cell culture liquid/gas-phase chamber 20 of FIGS. 2A-2C, wherein differentiated (Diff) and undifferentiated (Undiff) cultures of CF cells were incubated with or without 100 ng/ml of IFN-γ for 19-20 hours and the level of NO in the airspace above the cultures was measured, and where the baseline level of gNO was low in differentiated cultures, but was stimulated to high levels in differentiated cultures treated with IFN-γ, and where levels of gNO were low in undifferentiated cultures with or without IFN-γ treatment;

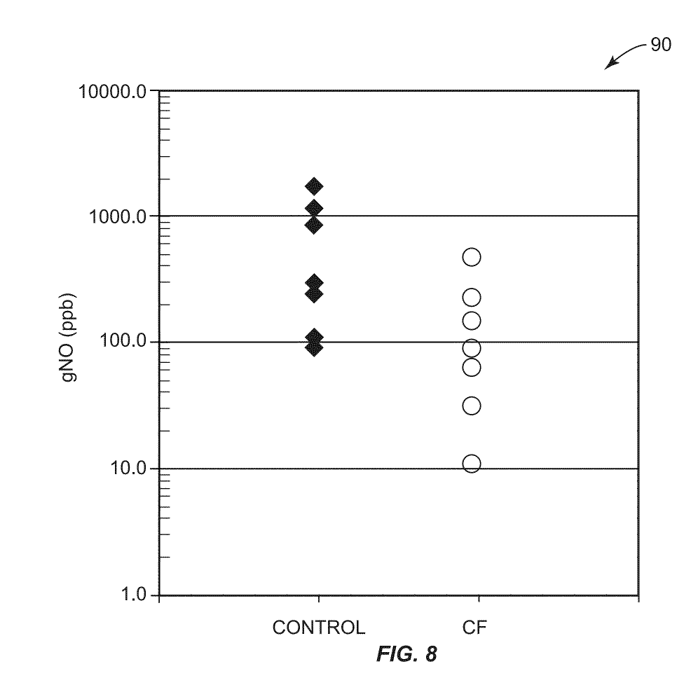

When stimulated with IFN-γ, CF cells responded with an increase in iNOS expression (see FIG. 6) and a robust increase in gNO levels, averaging 192.3+/−68.9 ppb (n=21; 8 donors; significantly different at p<0.0002). However, under both conditions, the level of gNO produced by the CF cells was lower than that produced by normal cultures. Differentiated cultures of normal HBE cells produced about twice as much gNO as CF cells under baseline conditions (10.9 vs. 5.8 ppb), and when stimulated with IFN-γ averaged ˜3-fold higher levels of gNO than differentiated cultures of CF cells treated with IFN-γ (576 vs. 192 ppb; significant at p=0.03). As observed for measurements of exhaled NO directly from patients, there was a large overlap between the levels of gNO from normal and CF cultures. This is illustrated in the chart 90 in FIG. 8, which is an exemplary chart showing gas-phase NO levels from control and CF HBE cell cultures. FIG. 8 shows the average values obtained for cultures from each donor.

Similarly to undifferentiated control cells, undifferentiated CF cells exhibited low levels of gNO (see FIG. 7), both in the basal state (5.6+/−2.2 ppb; n=12, 4 donors) and after stimulation with IFN-γ (15.6+/−6.8 ppb; n=9, 3 donors). These results were not significantly different from each other, nor were the levels obtained significantly different from those of undifferentiated normal cells. Submersion of CF cultures also greatly reduced the amount of gNO, with differentiated CF cells stimulated with IFN-γ in these experiments averaging 98+/−35 ppb, compared to only 11+/−4.3 ppb when submerged with 420 μl of PBS (p=0.003; n=9; 3 donors).

### Levels of Nitrate/Nitrite

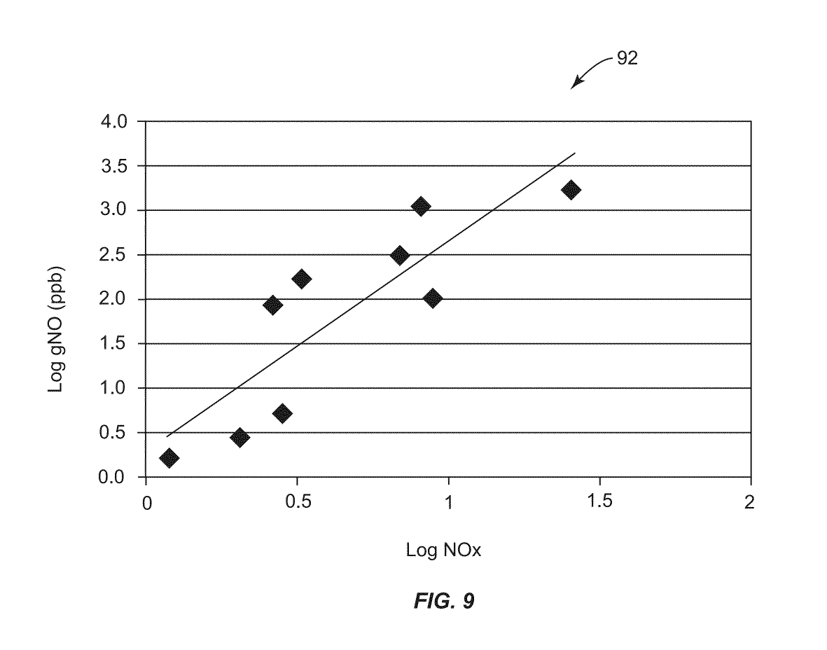

Nitric oxide present in biological fluids rapidly reacts with other molecules to produce a variety of products. In aqueous solutions, nitrite and nitrate are two of the major metabolites of NO reaction. To determine if the level of NO products in the basal media increased in a similar manner as the levels of gNO, the total amount of nitrate/nitrite (NOx) in a subset of experiments using the Griese reaction was measured. As expected, the level of nitrate/nitrite in the media was higher in cultures treated with IFN-γ than in untreated cultures, averaging 9.3+/−3.1 μM in treated cultures compared to 2.0+/−0.5 μM in untreated cultures (p=0.005; donors=7, 3). The level of NOx in the media increased proportionally to the level of gNO, with a correlation coefficient of 0.74 (y=2.33x+0.28; R²=0.74), as shown in the chart 92 in FIG. 9. FIG. 9 is an exemplary chart showing the correlation of gas-phase NO with NOx concentration in the basal media. Interestingly, in cultures submerged with 420 μl of PBS, the levels of nitrate/nitrite in the basal media were also lower than in cultures with only 50 μL on the apical surface (11.9+/−7.7 vs. 7+/−4.8 μM; p=0.031; donors=3), indicating that the presence of a thick ASL likely inhibited total cellular production of NO. Attempts to measure nitrate/nitrite directly in the apical fluid were unsuccessful; either because the levels of NO were below the limit of detection or because the presence of cellular produced factors (mucus, proteins, inhibitors) prevented the detection of NO metabolites in this compartment.

### Discussion

In these studies, the levels of gas-phase NO (gNO) in cultures of control and cystic fibrosis human bronchial and nasal epithelial cells under several conditions was measured. Suresh et al. showed that cultures of HBE cells grown at the air/liquid interface produced measurable amounts of gNO, and that the level of iNOS and gNO could be stimulated by IL-13 (21). Our data extend their observations to a larger number of samples and conditions, and further, compares the level of gNO produced by CF cells to a control group of non-CF samples. In agreement with their studies, it was found that un-stimulated HBE cells produced very low levels of gas phase NO under our standard conditions, but this could be increased ˜50 fold by treatment with IFN-γ. This result is consistent with previous studies that have demonstrated the induction of iNOS by IFN-γ (7-10). It was also observed that a wide range of NO production between samples from different individuals. This variation was also observed in the study published by Suresh et al. (21), although they studied a much smaller number of samples (3). Importantly, such variation is frequently observed in measurements of exhaled NO directly from individuals, even in selected control groups (17). Thus it seems that the level of NO production is influenced by genetic factors, and the influence that these factors exert appears to be maintained in vitro.

One advantage of culturing airway epithelial cells (nasal and bronchial) at an air/liquid interface is that the cells undergo mucociliary differentiation and morphologically resemble the in vivo airway epithelium. To examine the effect of differentiation on gNO levels, gNO levels from undifferentiated cultures of HBE cells, with and without stimulation by IFN-γ were measured. Under baseline conditions, undifferentiated cultures produced lower levels of gNO than differentiated cultures, although the levels of gNO under both conditions were very low (<20 ppb), and sometimes difficult to distinguish from baseline. However, this difference was much greater when cultures were treated with IFN-γ. Undifferentiated cultures treated with IFN-γ showed only an approximate 2-fold increase in gNO (˜10 ppb) compared to the large increase observed in differentiated cultures (>500 ppb). This data demonstrates that the response of HBE cells to IFN-γ, and subsequently the level of gNO, is dependent on the level of differentiation, and suggests that use of well-differentiated air/liquid interface cultures of HBE cells may be preferable to undifferentiated cell lines for studies of the regulation of NO synthesis.

Differentiated cultures of HBE cells produce and secrete mucus on their apical surface and the effect of different depths of apical fluid on gNO levels was also investigated. Surprisingly, the accumulation of gNO was drastically reduced from cultures incubated with relatively small volumes of PBS on their surface. Adding 420 μL to the surface of a 4.2 cm² culture (a predicted depth of only 1 mm) reduced the average gNO concentration in IFN-γ stimulated cultures >60-fold. A study by Worlitzsch (24) et al. demonstrated that under similar “thick film” conditions the oxygen partial pressure in ASL fluid decreases with increasing depth resulting in a hypoxic state. Because oxygen is an essential substrate for the production of NO, the extremely low levels of gNO released by cultures under thick film conditions may be the result of limited availability of O₂. Importantly, the pO₂ in mucopurulent material in CF airways was found to be close to zero (24). Thus the low levels of exhaled NO in CF patients may be, in part, due to hypoxic conditions near the sites of infection/inflammation. Similarly, patients with primary ciliary dyskinesia who have a genetic defect that impairs ciliary function and causes mucus accumulation also exhibit extremely low levels of nNO.

One of the goals of the current study was to determine if the reported low levels of NO in exhaled air from CF patients would be reproduced by well-differentiated cultures of airway epithelial cells, indicating that the low levels of NO were a direct effect of the absence of functional CFTR in airway epithelial cells. In these studies, CF cells stimulated with IFN-γ averaged 3-fold less NO than control cells, however it was observed that substantial overlap between the control and CF groups (see FIG. 8). Because the cells used in this study were obtained from donor tissue as it became available, it was not possible to carefully match the control and CF cells for such factors as age, sex, and smoking history. Nevertheless, in these studies the levels of gNO measured directly from well-differentiated cultures of HBE cells reproduced the CF phenotype observed in vivo, demonstrating that this model system at least partially reproduces the in vivo observations, and will be useful for further studies. Additional studies will be necessary to fully understand how the absence of CFTR affects the level of gNO production from airway epithelial cells, and the role decreased levels of NO may play in CF disease pathogenesis.

The chamber devices discussed herein were developed to study release of nitric oxide (NO) by cultured healthy, cystic fibrosis (CF) and primary ciliary dyskinesia (PCD) airways epithelial cells into the headspace. NO in the airways has many important functions including broncho- and vaso-dilation, control of ciliary motility and others. In diseased state the production on NO can be decreased (CF, PCD) or increased (airways inflammation). However, the mechanisms responsible for the low levels of NO in e.g., PCD have not been identified. Studying signaling pathways, immune responses or other physio- or patho-physiological functions of nitric oxide synthase (NOS) activity and NO metabolites produced by cell cultures in response to various stimuli has been typically assessed by measuring NO-related species in cultured cells media. This approach, however, does not account for an unbound NO released by cells directly into lumen, the gas headspace. Direct measurement of NO in nasal cavity or the bronchial tree shows that the amount of released NO by, e.g., airway epithelial cells as gaseous NO can be substantial. Repeatedly high production of gaseous NO by airway epithelial cells has been measured. Actually, the amount of produced NO has almost a diagnostic function. The release of NO into airways as a gas reflects the regulation and metabolism of these cells. Therefore, when studied as cell cultures it is important to study their function in both the liquid and the gas-phase as well. The exemplary chamber device 20 herein in FIGS. 2A-2C allows investigating such mechanisms and potential therapeutic approaches.

Many modifications and other embodiments of the embodiments set forth herein will come to mind to one skilled in the art to which the embodiments pertain having the benefit of the teachings presented in the foregoing descriptions and the associated drawings.

Therefore, it is to be understood that the description and claims are not to be limited to the specific embodiments disclosed and that modifications and other embodiments are intended to be included within the scope of the appended claims. It is intended that the embodiments cover the modifications and variations of the embodiments provided they come within the scope of the appended claims and their equivalents. Although specific terms are employed herein, they are used in a generic and descriptive sense only and not for purposes of limitation.

### REFERENCES ADDENDUM

The following references are all incorporated herein in the present application by reference in their entireties:
1. Ricciardolo F L, Sterk P J, Gaston B, Folkerts G. Nitric oxide in health and disease of the respiratory system. Physiol Rev. 2004; 84(3):731-65.
2. Zhan X, Li D, Johns R A. Expression of endothelial nitric oxide synthase in ciliated epithelia of rats. J Histochem Cytochem. 2003; 51(1):81-7.
3. Jain B, Rubinstein I, Robbins R A, Leise K L, Sisson J H. Modulation of airway epithelial cell ciliary beat frequency by nitric oxide. Biochem Biophys Res Commun. 1993; 191(1):83-8.
4. Jain B, Rubinstein I, Robbins R A, Sisson J H. TNF-alpha and IL-1 beta upregulate nitric oxide-dependent ciliary motility in bovine airway epithelium. Am J. Physiol. 1995; 268(6 Pt 1):L911-7.
5. Li D, Shirakami G, Zhan X, Johns R A. Regulation of ciliary beat frequency by the nitric oxide-cyclic guanosine monophosphate signaling pathway in rat airway epithelial cells. Am J Respir Cell Mol. Biol. 2000; 23(2):175-81.
6. Kharitonov S A, Barnes P J. Exhaled biomarkers. Chest. 2006; 130(5):1541-6.
7. Asano K, Chee C B, Gaston B, Lilly C M, Gerard C, Drazen J M, et al. Constitutive and inducible nitric oxide synthase gene expression, regulation, and activity in human lung epithelial cells. Proc Natl Acad Sci USA. 1994; 91(21):10089-93. PMCID: 44963.
8. Jiang J, Malavia N, Suresh V, George S C. Nitric oxide gas phase release in human small airway epithelial cells. Respir Res. 2009; 10:3. PMCID: 2633284.
9. Nathan C, Xie Q W. Regulation of biosynthesis of nitric oxide. J Biol Chem. 1994; 269(19):13725-8.
10. Salkowski C A, Detore G, McNally R, van Rooijen N, Vogel S N. Regulation of inducible nitric oxide synthase messenger RNA expression and nitric oxide production by lipopolysaccharide in vivo: the roles of macrophages, endogenous IFN-gamma, and TNF receptor-1-mediated signaling. J. Immunol. 1997; 158(2):905-12.
11. Bush A, Chodhari R, Collins N, Copeland F, Hall P, Harcourt J, et al. Primary ciliary dyskinesia: current state of the art. Arch Dis Child. 2007; 92(12):1136-40.
12. Leigh M W, Pittman J E, Carson J L, Ferkol T W, Dell S D, Davis S D, et al. Clinical and genetic aspects of primary ciliary dyskinesia/Kartagener syndrome. Genet Med. 2009; 11(7):473-87.
13. Lundberg J O, Weitzberg E, Nordvall S L, Kuylenstierna R, Lundberg J M, Alving K. Primarily nasal origin of exhaled nitric oxide and absence in Kartagener's syndrome. Eur Respir J. 1994; 7(8):1501-4.
14. Noone P G, Leigh M W, Sannuti A, Minnix S L, Carson J L, Hazucha M, et al. Primary ciliary dyskinesia: diagnostic and phenotypic features. Am J Respir Crit Care Med. 2004; 169(4):459-67.
15. Leigh M W, O'Callaghan C, Knowles M R. The challenges of diagnosing primary ciliary dyskinesia. Proc Am Thorac Soc. 2011; 8(5):434-7. PMCID: 3209576.
16. Balfour-Lynn I M, Layerty A, Dinwiddie R. Reduced upper airway nitric oxide in cystic fibrosis. Arch Dis Child. 1996; 75(4):319-22.
17. Grasemann H, Michler E, Wallot M, Ratjen F. Decreased concentration of exhaled nitric oxide (NO) in patients with cystic fibrosis. Pediatr Pulmonol. 1997; 24(3): 173-7.
18. Lundberg J O, Nordvall S L, Weitzberg E, Kollberg H, Alving K. Exhaled nitric oxide in paediatric asthma and cystic fibrosis. Arch Dis Child. 1996; 75(4):323-6.
19. Meng Q H, Polak J M, Edgar A J, Chacon M R, Evans T J, Gruenert D C, et al. Neutrophils enhance expression of inducible nitric oxide synthase in human normal but not cystic fibrosis bronchial epithelial cells. J. Pathol. 2000; 190(2):126-32.
20. Steagall W K, Elmer H L, Brady K G, Kelley T J. Cystic fibrosis transmembrane conductance regulator-dependent regulation of epithelial inducible nitric oxide synthase expression. Am J Respir Cell Mol. Biol. 2000; 22(1):45-50.
21. Suresh V, Mih J D, George S C. Measurement of IL-13-induced iNOS-derived gas phase nitric oxide in human bronchial epithelial cells. Am J Respir Cell Mol. Biol. 2007; 37(1):97-104.
22. Thomas S R, Kharitonov S A, Scott S F, Hodson M E, Barnes P J. Nasal and exhaled nitric oxide is reduced in adult patients with cystic fibrosis and does not correlate with cystic fibrosis genotype. Chest. 2000; 117(4):1085-9.
23. Fulcher M L, Gabriel S, Burns K A, Yankaskas J R, Randell S H. Well-differentiated human airway epithelial cell cultures. Methods Mol Med. 2005; 107:183-206.
24. Worlitzsch D, Tarran R, Ulrich M, Schwab U, Cekici A, Meyer K C, et al. Effects of reduced mucus oxygen concentration in airway Pseudomonas infections of cystic fibrosis patients. J Clin Invest. 2002; 109(3):317-25.

We claim:

1. A liquid/gas-phase chamber device for producing gas-phase and liquid-phase components released by cultured cells in response to stimuli, comprising:
a chamber body forming an internal chamber;
an air-liquid partition disposed in the internal chamber configured to interactively separate the internal chamber into a liquid compartment and a gas compartment;
at least one transwell disposed through the air-liquid partition, and at least one transwell configured to support at least one cell culture;
the at least one transwell comprising a first end disposed in the liquid compartment of the chamber body to capture a release of metabolic products from the cells of the at least one cell culture in liquid disposed in the liquid compartment; and
the at least one transwell further comprising a second end disposed in the gas compartment of the internal chamber to capture a release of gas by the cells of the at least one cell culture.

2. The liquid/gas-phase chamber device of claim 1, wherein the gas compartment is provided in a headspace of the internal chamber of the chamber body.

3. The liquid/gas-phase chamber device of claim 1, further comprising at least one port disposed in the chamber body, the at least one port configured to receive an injection of material to be disposed in the internal chamber and allow an extraction of material disposed in the internal chamber.

4. The liquid/gas-phase chamber device of claim 3, wherein the at least one port is disposed in the chamber body and fluidly coupled to the gas compartment of the internal chamber of the chamber body.

5. The liquid/gas-phase chamber device of claim 3, wherein the at least one port is disposed in the chamber body and fluidly coupled to the liquid compartment of the internal chamber of the chamber body.

6. The liquid/gas-phase chamber device of claim 1, wherein the chamber body is formed from Teflon®.

7. The liquid/gas-phase chamber device of claim 1, further comprising a lid configured to be received by a top portion of the chamber body forming at least a portion of the gas compartment of the internal chamber to provide an air-tight chamber body.

8. The liquid/gas-phase chamber device of claim 1, further comprising a first valve in fluid contact with the gas compartment of the internal chamber of the chamber body, and a second valve in fluid contact with the gas compartment of the internal chamber of the chamber body;
the first valve and the second valve each configured to be opened to allow ingress and egress of air into and from the gas compartment, and the first valve and the second valve each configured to be closed to preventingress and egress of air into and from the gas compartment.

9. The liquid/gas-phase chamber device of claim 8, wherein at least one of the first valve and the second valve comprises a 3-way valve comprising a first open position, a second open position, and a closed position.

10. The liquid/gas-phase chamber device of claim 1, wherein the at least one transwell further comprises at least one membrane disposed in the first end of the at least one transwell, the at least one membrane configured to be a disposed in the liquid compartment of the internal chamber of the chamber body to capture the release of the metabolic products from the cultured cells of the at least one cell culture in the liquid disposed in the liquid chamber.

11. A method of producing gas-phase component releases of cultured cells in response to stimuli, comprising:
transferring at least one transwell containing cultured cells from at least one cultured cell growth tray to an air-liquid partition for a liquid/gas-phase chamber device;
disposing stimuli liquid into a liquid compartment of an interior chamber of a chamber body of the liquid/gas-phase chamber device formed by the air-liquid partition disposed in an internal chamber of the chamber body the air-liquid partition configured to interactively separate the internal chamber into the liquid compartment and a gas compartment;
inserting the air-liquid partition with the at least one transwell disposed therein into the internal chamber of the chamber body to place at least one membrane of the at least one transwell in contact with the stimuli liquid disposed in the liquid compartment of the internal chamber of the chamber body;
sealing the internal chamber with a lid received by a top portion of the chamber body forming at least a portion of the gas compartment of the internal chamber to provide an air-tight chamber body;
closing a first valve in fluid contact with the gas compartment of the internal chamber of the chamber body, and a second valve in fluid contact with the gas compartment of the internal chamber of the chamber body; and
incubating the cultured cells in the sealed internal chamber of the sealed chamber body for a defined period of time to allow the cultured cells to release a metabolic product in the stimuli liquid in the liquid compartment and release a metabolic product in the gas compartment, in response to exposure of the cultured cells to the stimuli liquid.

12. The method of claim 11, further comprising:
opening the first valve and the second valve;
directing gas formed in the gas compartment of the internal chamber of the sealed chamber body to an analyzer; and
analyzing at least one gas-phase component of the gas formed in the gas compartment of the internal chamber of the sealed chamber body.

13. The method of claim 12, further comprising measuring an amount of the at least one gas-phase component of the gas formed in the gas compartment.

14. The method of claim 12, further comprising measuring a production rate of the at least one gas-phase component of the gas formed in the gas compartment.

15. The method of claim 12, wherein the at least one gas-phase component comprises a gas-phase Nitric Oxide (NO) release in the gas compartment as a result of the incubating of the cultured cells.

16. The method of claim 15, wherein the cultured cells are comprised of CF human bronchial epithelial (HBE) cells.

17. The method of claim 12, further comprising closing the first valve and the second valve after opening the first valve and the second valve, directing the gas formed in the gas compartment of the internal chamber of the sealed chamber body to an analyzer, and analyzing the gas formed in the gas compartment of the internal chamber of the sealed chamber body.

18. The method of claim 12, further comprising flushing the gas compartment after the analyzing of the at least one gas-phase component of the gas formed in the gas compartment of the internal chamber of the sealed chamber body.

19. The method of claim 17, further comprising reanalyzing the at least one gas-phase component of the gas formed in the gas compartment of the internal chamber of the sealed chamber body after the flushing.

20. The method of claim 11, further comprising extracting at least a portion of the stimuli liquid through at least one port disposed in the chamber body and fluidly coupled to the liquid compartment of the internal chamber of the chamber body.

21. The method of claim 11, further comprising injecting a material through at least one port disposed in the chamber body and fluidly coupled to the liquid compartment of the internal chamber of the chamber body to dispose the material in the liquid compartment of the internal chamber of the chamber body.

22. The method of claim 11, further comprising injecting a material through at least one port disposed in the chamber body and fluidly coupled to the gas compartment of the internal chamber of the chamber body to dispose the material in the gas compartment of the internal chamber of the chamber body.
